(19) United States
(12) Patent Application Publication
(10) Pub. No.: US 2022/0202434 A1
(43) Pub. Date: Jun. 30, 2022

(54) APPARATUS, SYSTEMS AND METHODS FOR ERGONOMIC MEDICAL PROCEDURES

(71) Applicant: University of Iowa Research Foundation, Iowa City, IA (US)

(72) Inventor: Evgeny Arshava, Iowa City, IA (US)

(21) Appl. No.: 17/563,929

(22) Filed: Dec. 28, 2021

Related U.S. Application Data
(60) Provisional application No. 63/131,058, filed on Dec. 28, 2020.

(51) Int. Cl. A61B 17/30

(52) U.S. Cl. CPC A61B 17/30; A61B 2017/00424

(57) ABSTRACT

Ergonomic support units for use with medical instruments, such as surgical forceps. In various implementations the support units may include wings, rings, winged rings, or other support structures. An ergonomic instrument including a first support unit shaped to support at least one finger of a user and a second support unit shaped to support a thumb of a user, wherein the first support unit and second support unit are configured to reduce effort required to manipulate the ergonomic instrument.

### CROSS-REFERENCE TO RELATED APPLICATION(S)

This application claims the benefit under 35 U.S.C. § 119(e) to U.S. Provisional Application 63/131,058, filed Dec. 28, 2020, and entitled Apparatus, Systems and Methods for Ergonomic Medical Procedures, which is hereby incorporated herein by reference in its entirety for all purposes.

### TECHNICAL FIELD

The disclosed technology relates generally to medical devices and in particular, to the devices, methods, and design principles allowing for the precise, stable, ergonomic use of forceps and other instruments in medical procedures. This has implications for medical procedures as well as other applications.

### BACKGROUND

Surgical instruments are constantly evolving along with surgical techniques. While various minimally invasive approaches, such as thoracoscopy, laparoscopy, robotic assisted operations, are gaining popularity, open operations remain the foundation of surgery in the U.S. and worldwide.

During surgical operations various hand-held instruments are used to perform different manipulations, for example, holding tissues (clamps and forceps), separating tissues (scalpels, scissors and dissectors), retracting tissues (retractor), suturing (needle holders), and others as would be appreciated. Multiple types of forceps that vary in length, shape of tip, and shape of handle currently exist, as is shown in FIG. 1. With use of known forceps, and particularly forceps with extended length, certain limitations of their use are observed.

There is a need in the art for improved ergonomic devices and associated systems and methods for performing surgery using handheld tools.

### BRIEF SUMMARY

Discussed herein are various devices, systems, and methods relating to handheld forceps and other instruments having support units, such as rings, wings and/or other ergonomic portions on one or both handles/arms. Depending on the instrument and its length, the exact location of the support unit(s) relative to the end of the instrument, and the angle of attachment to the axis of the handle may vary. Additionally, the position of the support units may vary for right and left-handed surgeons, as would be appreciated. Yet, in certain implementations the support units are ambidextrous, that is, able to be used by both left and right hands without reconfiguration.

According to various aspects, the various implementations of the device is ergonomic and improves the stability of the instrument in a user's hands. Further, the various implementations may allow for a decrease in effort necessary to hold the instrument. In various implementations, the support units can be permanently attached to the instrument, for example the support units may be molded with the instrument during original manufacturing process. Optionally, the support units may be detachable, such that the support units can be adjustability secured on and removed from the instrument per the user's preference or the use case.

Example 1 relates to an ergonomic forceps comprising a first support unit shaped to support at least one finger of a user and a second support unit shaped to support a thumb of a user, wherein the first support unit and second support unit are configured to reduce effort required to manipulate the ergonomic forceps.

In Example 2, the forceps of Example 1, wherein the first support unit and second support unit are integral to the ergonomic forceps.

In Example 3, the forceps of any of Examples 1-2, wherein the first support unit and second support unit are removable from the ergonomic forceps.

In Example 4, the forceps of any of Examples 1-3, wherein the first support unit and the second support unit each comprise an instrument engaging portion for engagement with an arm of the ergonomic forceps.

In Example 5, the forceps of any of Examples 1-4, wherein the instrument engaging portion defines a wholly or partially enclosed lumen, wherein the arm of the ergonomic forceps is inserted into the lumen.

In Example 6, the forceps of any of Examples 1-5, wherein the instrument engaging portion is a clip.

In Example 7, the forceps of any of Examples 1-6, wherein the first support unit is a winged support unit comprising a first curve defining a first seat for supporting a first finger of the user and a second curve defining a second seat for supporting a second finger of the user.

In Example 8, the forceps of any of Examples 1-7, wherein the first support unit further comprises a central ridge between the first curve and second curve for supporting a third finger of the user.

In Example 9, the forceps of any of Example 1-8, wherein the second support unit is a winged support unit comprising a curve defining a seat for supporting the thumb of the user.

In Example 10, the forceps of any of Examples 1-9, wherein the second support unit further comprises a ridge.

In Example 11, the forceps of any of Examples 1-10, wherein the first support unit is a ringed support unit comprising at least one ring.

In Example 12, the forceps of any of Examples 1-11, wherein the first support unit comprises two rings.

In Example 13, the forceps of any of Examples 1-13, wherein the second support unit is a ring.

Example 14 relates to a set of support units for a medical instrument comprising a first support unit shaped to support at least one finger of a user, the first support unit comprising a first instrument engaging portion configured to be engaged with a first arm of the medical instrument and a second support unit shaped to support a thumb of a user, the second support unit comprising a second instrument engaging portion configured to be engaged with a second arm of the medical instrument.

In Example 15, the support units of Example 14, wherein the first support unit comprises two rings and wherein the second support unit comprises one ring.

In Example 16, the support units of any of Examples 14-15, wherein the first support unit comprises two curves defining two seats and wherein the second support unit comprises one curve defining one seat.

In Example 17, the support units of any of Examples 14-16, wherein the first instrument engaging portion is a clip and wherein the second engagement portion is a clip.

In Example 18, the support units of any of Examples 14-17, wherein the first support unit and second support unit are selectively removable from the medical instrument.

In Example 19, the support units of any of Examples 14-19, wherein the medical instrument is forceps with exchangeable tips.

Example 20 relates to an ergonomic forceps supports comprising a first winged support comprising a first curve defining a first seat; a second curve defining a second seat; a first ridge disposed between the first curve and the second curve; and a first instrument engaging portion shaped for engagement with a first arm of an instrument and a second winged support unit comprising: a third curve defining a third seat; a second ridge disposed centrally on the third curve; and a second instrument engaging portion shaped for engagement with a second arm of the instrument.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows various known handheld forceps.

FIG. 2A shows a side view of winged forceps, according to one implementation.

FIG. 2B shows a top view of winged forceps, according to one implementation.

FIG. 3A shows a perspective view of winged forceps in use, according to one implementation.

FIG. 3B shows a side view of winged forceps in use, according to one implementation.

FIG. 4A shows a side view of ringed forceps, according to one implementation.

FIG. 4B shows a bottom view of ringed forceps, according to one implementation.

FIG. 5A shows a side view of ringed forceps in use, according to one implementation.

FIG. 5B shows a top view of ringed forceps in use, according to one implementation.

FIG. 6A shows a side view of finger rings, according to one implementation.

FIG. 6B shows a front view of finger rings, according to one implementation.

FIG. 6C shows a top view of finger rings, according to one implementation.

FIG. 6D shows a bottom view of finger rings, according to one implementation.

FIG. 6E shows a perspective view of finger rings, according to one implementation.

FIG. 7A shows a side view of a thumb ring, according to one implementation.

FIG. 7B shows a front view of a thumb ring, according to one implementation.

FIG. 7C shows a top view of a thumb ring, according to one implementation.

FIG. 7D shows a bottom view of a thumb ring, according to one implementation.

FIG. 7E shows a perspective view of a thumb ring, according to one implementation.

FIG. 8A shows a perspective view of finger rings, according to one implementation.

FIG. 8B shows a top view of finger rings, according to one implementation.

FIG. 9A shows a perspective view of a clip thumb ring, according to one implementation.

FIG. 9B shows a side view of a clip thumb ring, according to one implementation.

FIG. 9C is a side view of a clip, according to one implementation.

FIG. 10A is a perspective view of a split ring, according to one implementation.

FIG. 10B is a top view of a split ring, according to one implementation.

FIG. 11 is a perspective view of finger and thumb rings in use, according to one implementation.

FIG. 12A is a side view of winged supports, according to one implementation.

FIG. 12B is a side view of winged supports, according to one implementation.

FIG. 12C is a side view of winged supports, according to one implementation.

FIG. 12D is a side view of winged supports, according to one implementation.

FIG. 12E is a side view of winged supports, according to one implementation.

FIG. 12F is a side view of winged supports, according to one implementation.

FIG. 13 is a perspective view of winged supports, according to one implementation.

FIG. 14 is a side view of ringed supports, according to one implementation.

FIG. 15A is a perspective view of winged supports engaged with forceps, according to one implementation.

FIG. 15B is a bottom-up view of winged supports engaged with forceps, according to one implementation.

FIG. 15C is a bottom-up view of winged supports engaged with forceps, according to one implementation.

FIG. 15D is a close-up view of winged finger supports engaged with forceps, according to one implementation.

FIG. 15E is a bottom-up view of winged supports engaged with forceps, according to one implementation.

FIG. 15F is a bottom-up view of winged supports engaged with forceps, according to one implementation.

FIG. 15G is a top view of winged supports engaged with forceps, according to one implementation.

FIG. 16A is a perspective view of a winged finger support and a thumb ring engaged with forceps, according to one implementation.

FIG. 16B is a perspective view of a winged finger support and a thumb ring engaged with forceps having detachable/exchangeable tip, according to one implementation.

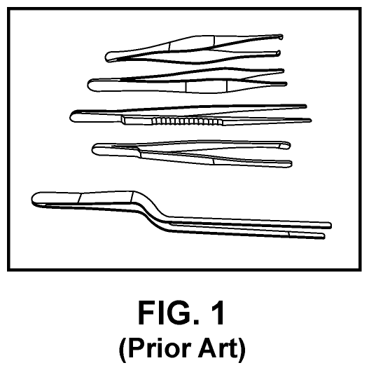

### DETAILED DESCRIPTION

Disclosed herein are various implementations of forceps and support devices for forceps and other surgical instruments. According to various aspects, the disclosed implementations are ergonomic and improve the stability of the instrument while in use. For example, during rotation prior known instruments tend to open to their normal shape and cause difficulty and stress on the hand. Further with extended or prolonged use, users can experience cramping sensations in their hand or forearm due to mechanical strain of holding an instrument. The various support devices, systems, and methods address these limitations and improve user experience and ergonomics.

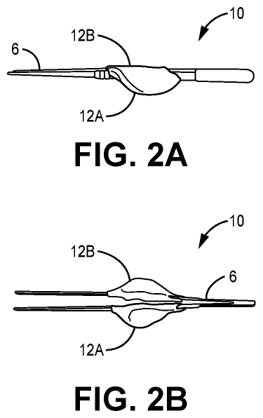

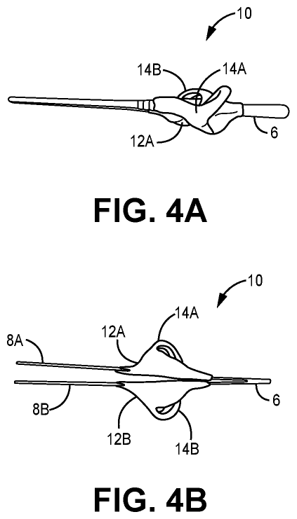

The various implementations disclosed or contemplated herein relate to instruments, such as but not limited to forceps 6 having one or more support unit(s) 10, such as, but not limited to, wings 12A, 12B, 21A, 21B (shown for example in FIGS. 2A-3B, 12A-F, 14, 16A-G and 17A-B), rings 14A-B and 18A-C (shown for example in FIGS. 4A-11, 15, and 17A-B), combinations thereof. The various support units 10 are constructed and arranged to provide support to the user's fingers and increase comfort and stability during use. It is appreciated that many configurations of support units 10 and instruments 6 are possible, including ambidextrous configurations.

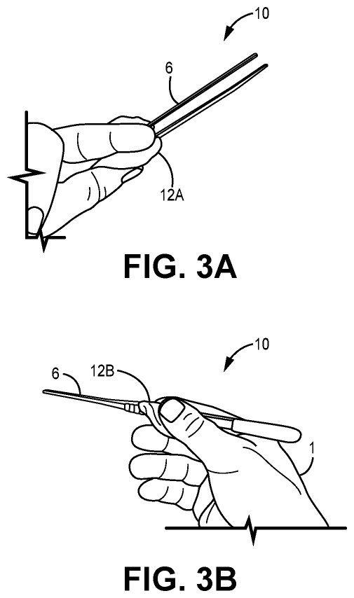

FIGS. 2A-3B depict implementations of forceps 6 with support units 10 in the form of ergonomic wings 12A, 12B. It would be understood that the wings 12A, 12B according to these implementations are constructed and arranged to allow for ease of grasping by the user's 1 forefinger and/or thumb, as well as cradling with the middle finger, as would be appreciated. Other implementations and configurations are of course possible.

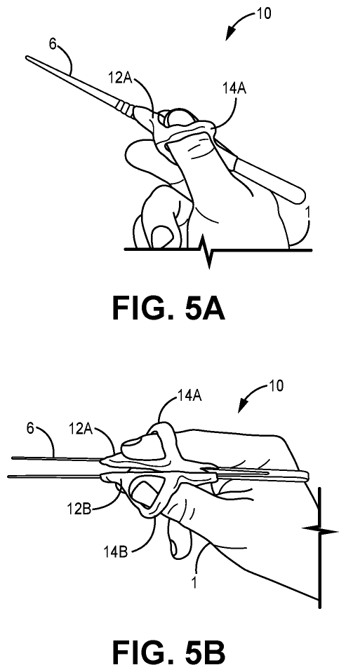

Turning now to the implementations of FIGS. 4A-5B, in these and other implementations, the forceps 6 and support units 10 can further include rings 14A, 14B disposed on one or both of the elongate arms 8A, 8B of the forceps 10. In certain implementations, the rings 14A, 14B are constructed and arranged to at least partially encircle a user's 1 finger(s)/thumb during use to create a secure hold. In certain implementations, the rings 14A, 14B may create support on all sides of a user's 1 finger(s)/thumb such that support is provided with any orientation of the user's fingers and hand during use. In various implementations, the rings 14A, 14B are integrated with ergonomic wings 12A, 12B, such that the wings 12A, 12B form one unit with the rings 14A, 14B on either or both arms 8A, 8B of the forceps 6.

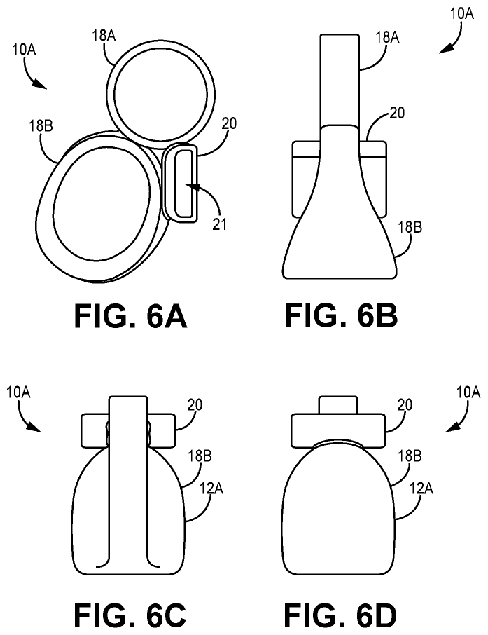

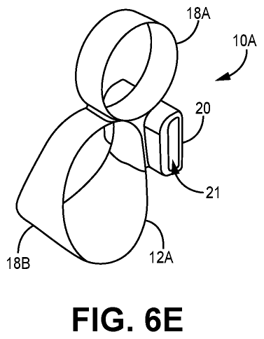

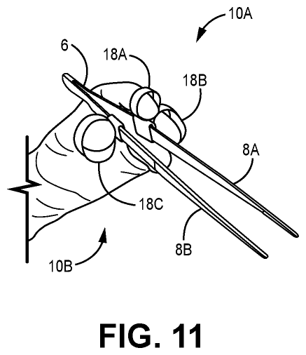

Turning now to FIGS. 6A-11, these implementations depict support units 10A, 20B having one or more rings 18A, 18B, 18C configured to be disposed on arms 8A, 8B of an instrument, such as the forceps 6 shown in FIG. 11.

Continuing with FIGS. 6A-11, in certain implementations, a first support unit 10A is configured to be disposed on a first arm 8A and a second support unit 10B is configured to be disposed on a second arm 8B substantially opposite the first arm 8A. Various other placements and configurations are possible for various instruments and uses, as would be appreciated.

The rings 18A, 18B, 18C may be in a variety of orientations in relation to the forceps 6 or other instrument. In certain implementations the some of the rings 18B, 18C are configured to be disposed adjacent to the arms 8A, 8B and another ring 18A disposed above the other rings 18B, 18C when the rings 18A, 18B, 18C are disposed on an instrument and the instrument is in a horizontal position, shown best in FIG. 11.

In one exemplary configuration, the first support unit 10A, shown in FIGS. 6A-E includes first and second rings 18A, 18B and an instrument engaging portion 20. In various implementations, the first ring 18A is shaped to be engaged with a user's index finger. In some implementations, the second ring 18B is shaped to be engaged with a user's middle and/or ring finger. Further, in certain implementations, the instrument engaging portion 20 defines a lumen 21, where one arm 8A, 8B of an instrument 6 can be inserted into the lumen 21 to hold or engage the support unit 10A on the instrument 6.

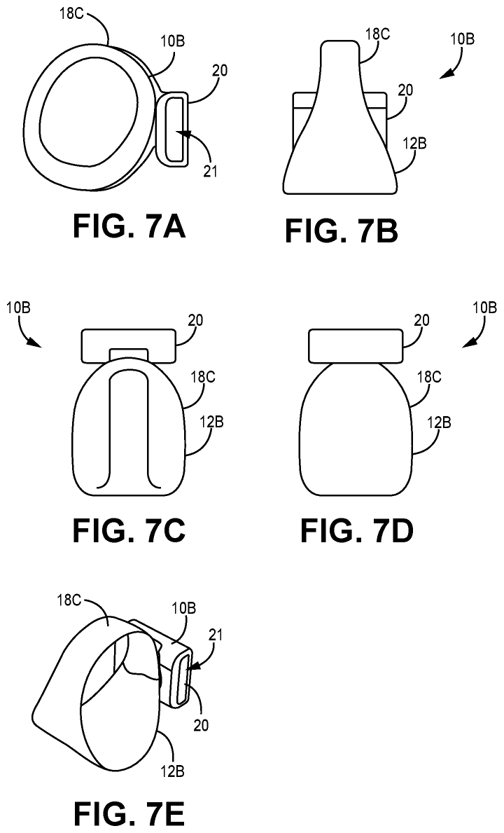

Turning now to FIGS. 7A-E, a second support unit 10B may include a third ring 16C. In various of these implementations, the third ring 18C is configured to support a user's thumb. The second support unit 10B may further include an instrument engaging portion 20 defining a lumen 21 shaped to hold and / or engage an instrument 6 such that the support unit 10C is held on the instrument 6 during use.

Various alternative configurations of the support units 10A, 10B of FIGS. 6A-7E are possible and would be apparent to one of skill in the art. Certain of the rings 18A, 18B, 18C according to these implementations may comprise wings 12A, 12B, as would be understood. That is, in various implementations, a portion of one or more rings 18A, 18B, 18C is flared or of extended width such as to constitute a wing 12A, 12B to support a user's digit. Further, the rings 18A, 18B, 18C may be configured to support a variety of user fingers and/or portions of a user's hand.

It is further appreciated that depending on the instrument 6 and its length, the exact location of the support units 10, 10A, 10B, and the rings 14A, 14B, 18A, 18B, 18C and/or wings 12A, 12B thereof, relative to the end of the instrument 6 may vary. Further, angle of attachment relative to axis of the instrument 6 may vary.

In certain implementations, the shape and orientation of the support units 10, 10A, 10B can be adjusted per user preference. That is, the support units 10, 10A, 10B can be slidably positioned more proximally or distally so as to accommodate user preference and/or the intended use. For example, the position of the rings 18A, 18B, 18C may vary for right and left-handed surgeons, as would be well-understood. Further, certain surgeons may prefer various grips or have differing sizes of hands requiring varying orientations and/or configuration of the support units 10, 10A, 10B. Alternatively, the support units 10, 10A, 10B may be ambidextrous allowing for use by both left and right-handed users.

Further, during use according to certain surgical procedures, one individual procedure may be best achieved by positioning the support units 10A, 10B in one position and a second individual procedure may be best achieved by positioning the support units 10A, 10B in a second position. Further, as would be readily appreciated, the user can make use of either hand or switch hands during a procedure, including while, for example, grasping tissue. According to certain implementations one or both of the support units 10A, 10B are capable of being selectively positioned proximately and/or distally along the arms 8A, 8B. Further orientations and uses are of course possible,

In alternate implementations, the support units 10, 10A, 10B can be permanently attached to the instrument 6 during original manufacturing process or at a later time. In alternate implementations, the support units 10, 10A, 10B are detachable such that the support units 10, 10A, 10B can be adjustability secured on and removed from the instrument 6 per the user's preference. In various implementations, the support units 10, 10A, 10B are removably securable to the instrument via the instrument engaging portion 20, shown variously in FIGS. 6A-11.

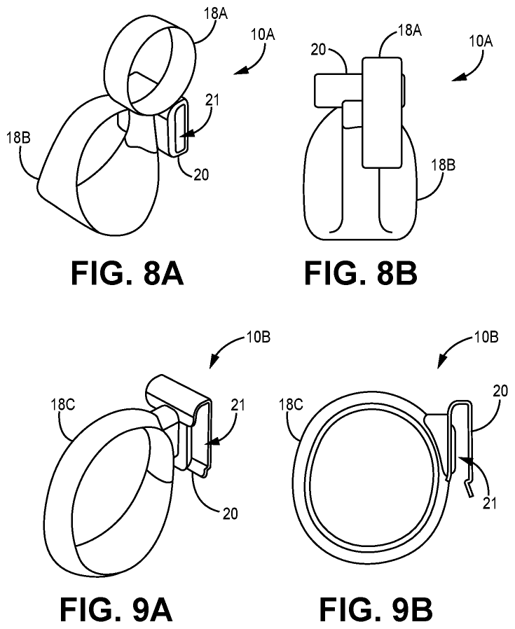

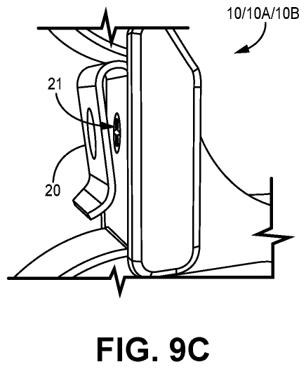

Turning to FIGS. 8A-B and 9A-C, the instrument engaging portion 20 may be a clip 20. In these and other implementations, the one or more of the support units 10A, 10B can be a selectively positioned on and/or detachable from an instrument 6 via a clip 20. Such an attachment portion 20 can define an enclosed slot or lumen 21 (as shown in FIGS. 6A-7E), or an open slot or lumen 21 (shown in FIGS. 9A-C and also shown various in figures herein and as will be discussed further below). Various alternative instrument engaging portion 19 configurations would be appreciated by those of skill in the art.

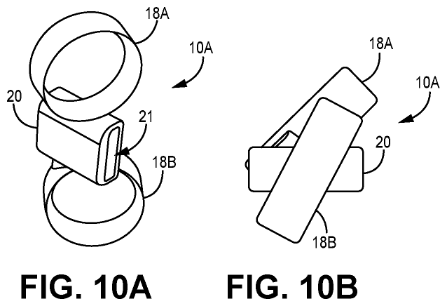

Turning now to FIGS. 10A-B, in various implementations, the rings 18A, 18B may be in a split-ring configuration, wherein the rings 18A, 18B on a first support unit 10A may be disposed at various angles about and relative to an arm 8A, 8B (shown in FIG. 11) of the instrument 6.

In certain implementations, the rings 18A, 18B are disposed on substantially opposite sides of the instrument engaging portion 20, and therefore are on substantially opposite sides of the instrument 6 when the support unit 10A is engaged with an instrument 6. In certain of these implementations, the rings 18A, 18B and the angle thereof are adjustable, such as via a rotatable movement. In alternative implementations, the rings 18A, 18B are in a fixed orientation.

Overall, the various designs and implementations provided herein are ergonomic, improve the stability of the instrument in a user's hands, and may allow for a decrease in the effort necessary to hold and use the instrument.

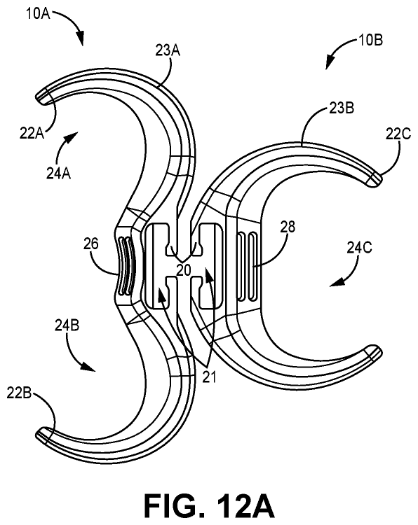

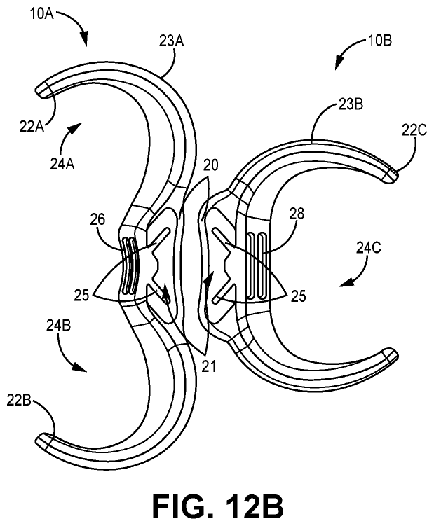

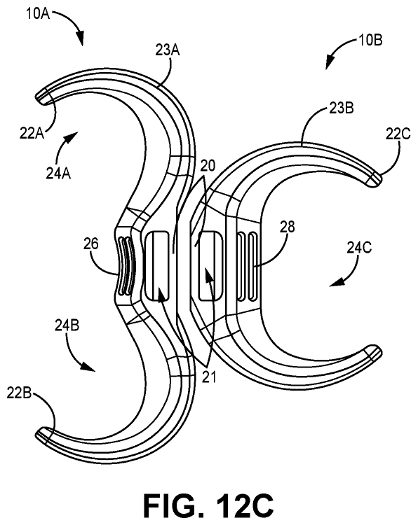

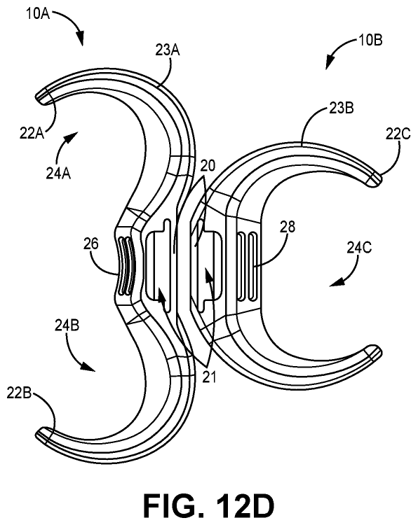

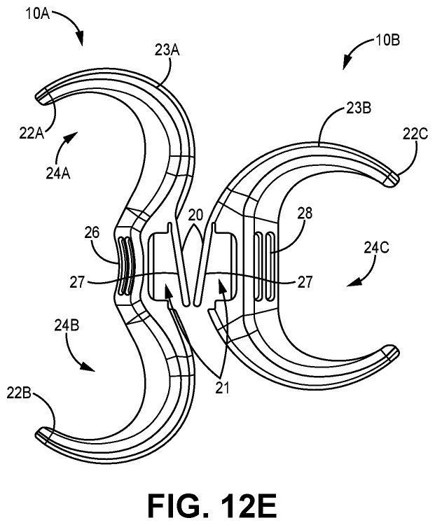

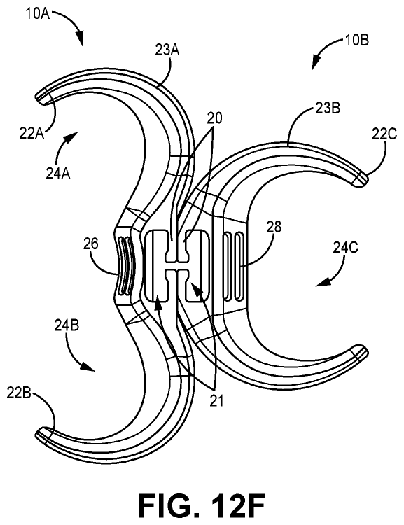

FIGS. 12A-16G show alternative implementations of the support units 10A, 10B and particularly support units 10A, 10B having wings 23A, 23B or rings 30A, 30B. As shown in FIGS. 12A-F and 15A-G, in various implementations, the support units 10A, 10B are constructed and arranged to be securable to and removable from an instrument 6 via the instrument engaging portion 20.

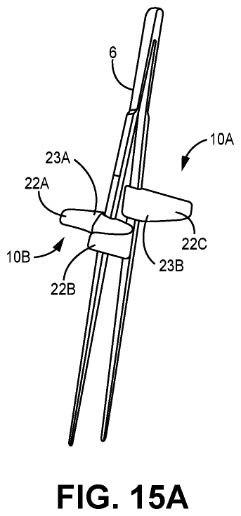

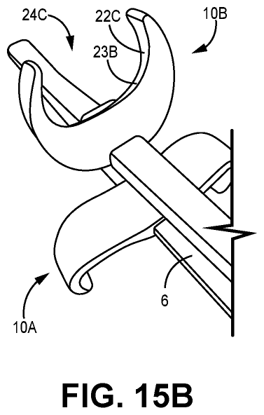

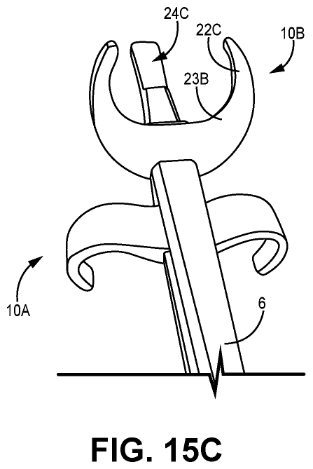

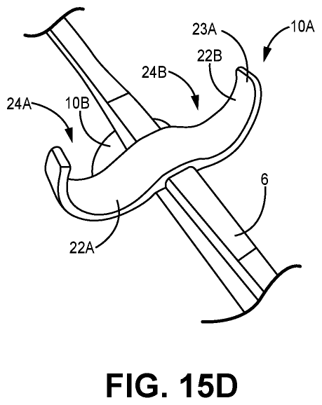

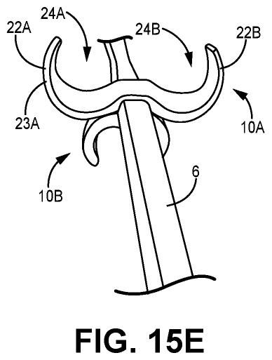

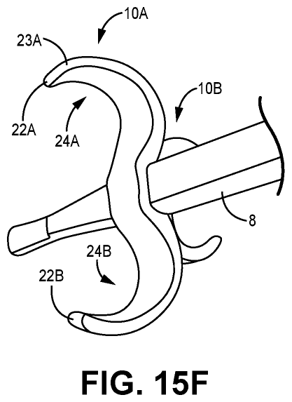

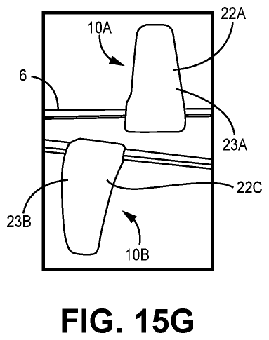

In certain implementations, the instrument engaging portion defines a lumen 21, in a first side of each of the wings 23A, 23B. In these implementations, the wings 23A, 23B can be placed on each arm 8A, 8B of the instrument 6 by placing a side of the instrument 6 through the lumen 21 until the wing 23A, 23B is secured on the instrument 10, as shown best in FIGS. 15A-G. In various implementations, the support units 10A, 10B are held on the instrument via the frictional forces between the instrument 6 and the instrument engaging portion 20.

In certain these implementations, the support units 10A, 10B can be removed by sliding each of the support units 10A, 10B along the arms 8A, 8B of the instrument 6 until the arms 8A, 8B are wholly outside of the lumen 21 and the instrument engaging units 20 are no longer engaged with the instrument 6, as would be understood.

The instrument engaging portion 20 may be in a variety of configurations, shown variously in FIGS. 12A-G. In FIG. 12A the instrument engaging portion 20 defines a partially enclosed lumen 21. In FIG. 12B the instrument engaging portion 20 defines a wholly enclosed lumen 21 with one or more projections 25 extending into the lumen 21. In these and other implementation the projects 25 are configured to engage the instrument 6 to increase friction and create a more secure hold. FIG. 12C shows an implementation where the instrument engaging portion 20 is a wholly enclosed lumen 21. FIG. 12D shows a further implementation where the instrument engaging portion 20 is a wholly enclosed lumen 21, where the lumen 21 has at least two widths to accommodate different sized/shaped instruments 6. FIG. 12E depicts a further implementations of the support units 10A, 10B where the instrument engaging portion 20 defines a lumen 21 and includes a hinged closure 27. A further implementation of a partially enclosed lumen 21 is shown in FIG. 12F.

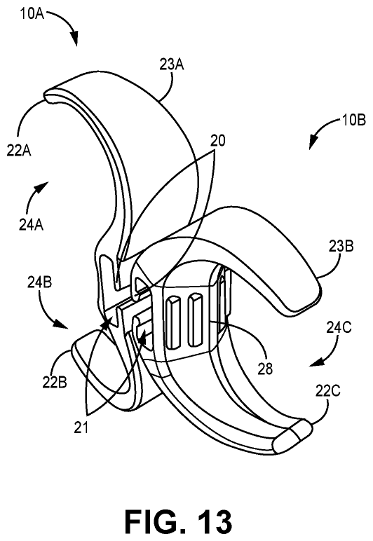

Continuing with the implementations of FIGS. 12A-12F and 13, in these and other implementations, support units 10A, 10B include one or more wings 23A, 23B. The wings 23A, 23B may define one or more curves 22A, 22B, 22C which in turn define one or more seats 24A, 24B, 24C. In various implementations, a first support unit 10A includes one wing 23A which defines curves 22A, 22B that in turn define seats 24A, 24B shaped to cradle a user's fingers. In some implementations, a first finger, such as a user's index finger, may be placed within a first seat 24A and a second finger, such as a user's middle or ring finger, may be placed within a second seat 24B.

In certain implementations, the support unit 10A includes a central ridge 26. In various of these implementations, the central ridge 26 is disposed substantially between two of the seats 24A, 24B and is configured to provide a contact point for a user's finger. For example, a user may place their index finger in a first seat 24A and their ring finger in the second seat 24B thereby resting their middle finger on the central ridge 26. In various implementations, the central ridge 26 may include one or more grooves to increase contact area and therefore friction along the central ridge 26 during use. As would be understood, in various procedures and in order to execute certain maneuvers this central ridge 26 provides necessary tactile feedback to a user and allows a user to exercise the necessary control over the instrument.

Continuing with FIGS. 12A-F and 13, in certain implementations, a second support unit 10B is provided. In various implementations, the second support unit 10B has a wing 23B defining at least one curve 22C which in turn defines a seat 24C. In certain implementations, the seat 24C is shaped to cradle a user's thumb during use. In certain implementations, the second support unit 10B includes a ridge 28, that may include one or more grooves. Similar to the central ridge 26 of the first support unit 10A, the ridge 28 may provide additional friction for a user to grip the support unit 10B and through that the instrument.

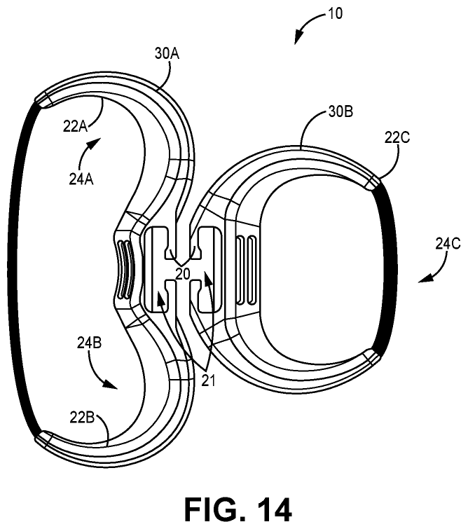

FIG. 14 shows an alternative implementation of the support units 10A, 10B. In this implementation, the support units 10A, 10B include enclosed rings 30A, 30B defining curves 22A, 22B, and 22C and in turn defining seats 24A, 24B, 24C similar to those described above.

Turning now to FIGS. 15A-15G, in various implementations, the support units 10A, 10B, wings 23A, 23B, curves 22A, 22B, 22C, and seats 24A, 24B, 24C are shaped to cradle various of a user's fingers in order to decrease the amount of muscle effort is required to operate the instrument 6, such as forceps 6 or other similar surgical instrument. In these implementations the support units 10A, 10B are ergonomic and ambidextrous. The support units 10A, 10B, the wings 23A, 23B and/or curves 22A, 22B, and 22C may also support a user's fingers during use. Further ergonomic features are of course possible and can be combined with any of the forgoing.

Continuing with FIGS. 15A-G, in these implementations, the individual support units 10A, 10B are symmetrical in design such that the support units 10A, 10B can be used be either left-handed, right-handed, or ambidextrous users or an instrument including the support units 10A, 10B can be passed between hands with necessitating changing or reorientation of the support units 10A, 10B. That is, the symmetrical design of the support units 10A, 10B, and particularly the first support unit 10A, allows for use by either the right hand or the left hand without need to replace or reorient the support units 10A, 10B on the instrument.

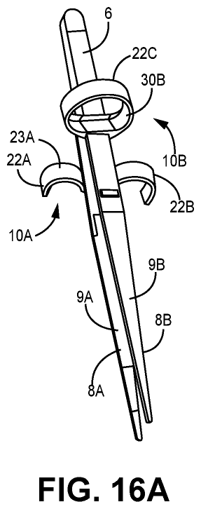

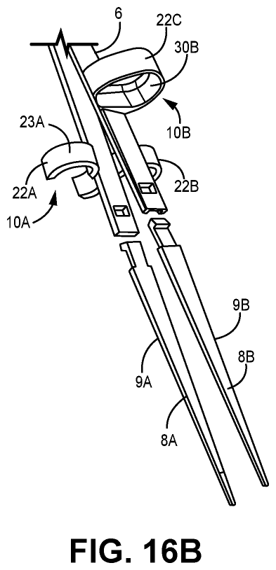

Turning to FIGS. 16A, 16B, in various implementations, the first support unit 10A is a winged 23A support while the second support 10B is a ringed 30B support. The alternate is also possible although not pictured. Further, FIGS. 16A, 16B depict forceps 6 with interchangeable/exchangable tips 9A, 9B. That is, the arms 8A, 8B of the forceps 6 have removeable tips 9A, 9B such that the type of forceps can be easily exchanged while maintaining the support units 10A, 10B on the upper portion of the forceps 6. Further in these implementations, the support units 10A, 10B may be integral to the upper portion of the forceps 6, as discussed above.

### EXAMPLES

Use of the forceps 6 with support units 10, 10A, 10B disclosed herein was studied using electromyography (“EMG”) to measure the amount of muscle effort exerted while using the forceps 6 with support units 10, 10A, 10B. In an initial study of more than 20 subjects, including surgeons and surgical trainees, surface EMG electrodes were applied to the thenar muscles and muscle activity was measured during various tests. Subjects used winged support units 10A, 10B of either standard or small size based on their hand size. The various tests included a peg board test, a repetitive pinch test, and a continuous pinch test.

In the peg board test, the use of forceps 6 with support units 10, 10A, 10B did not adversely affect performance (time and accuracy) compared to use of forceps with no support units. In the repetitive pinch test, there was significant evidence that participants had approximately 30% lower raw EMG values during rest periods, indicating less effort required to use the instrument with support units 10, 10A, 10B during the task. During continuous pinch tests there was better stability of the forceps 6 use (improved steadiness index) during task performance when using support units 10, 10A, 10B compared to not using support units.

Users were also asked to assess their subjective experience using the disclosed forceps 6 with support units 10, 10A, 10B using a post testing questionnaire. The subjective assessment/feedback of the device by users consistently demonstrated decreased fatigue and increased comfort during tests when using support units 10, 10A, 10B. The subjective reports correlated well with objective data of EMG, described above.

Over all, the results from the study showed a decrease in hand muscle effort as measured by EMG by about 30% when compared to use of forceps 6 without the disclosed support units 10A, 10B. Additionally increased comfort during manipulation was reported.

Although the disclosure has been described with reference to preferred embodiments, persons skilled in the art will recognize that changes may be made in form and detail without departing from the spirit and scope of the disclosed apparatus, systems and methods.

What is claimed is:

1. An ergonomic medical instrument comprising:
(a) a first support unit shaped to support at least one finger of a user and
(b) a second support unit shaped to support a thumb of a user,
wherein the first support unit and second support unit are configured to reduce effort required to manipulate the ergonomic medical instrument.

2. The ergonomic medical instrument of claim 1, wherein the first support unit and second support unit are integral to the ergonomic medical instrument.

3. The ergonomic medical instrument of claim 1, wherein the first support unit and second support unit are removable from the ergonomic medical instrument.

4. The ergonomic medical instrument of claim 1, wherein the first support unit and the second support unit each comprise an instrument engaging portion for engagement with an arm of the ergonomic medical instrument.

5. The ergonomic medical instrument of claim 4, wherein the instrument engaging portion defines a wholly or partially enclosed lumen, wherein the arm of the ergonomic medical instrument is inserted into the lumen.

6. The ergonomic medical instrument of claim 4, wherein the instrument engaging portion is a clip.

7. The ergonomic medical instrument of claim 1, wherein the first support unit is a winged support unit comprising:
(a) a first curve defining a first seat for supporting a first finger of the user and
(b) a second curve defining a second seat for supporting a second finger of the user.

8. The ergonomic medical instrument of claim 7, wherein the first support unit further comprises a central ridge between the first curve and second curve for supporting a third finger of the user.

9. The ergonomic medical instrument of claim 1, wherein the second support unit is a winged support unit comprising a curve defining a seat for supporting the thumb of the user.

10. The ergonomic medical instrument of claim 9, wherein the second support unit further comprises a ridge.

11. The ergonomic medical instrument of claim 1, wherein the first support unit is a ringed support unit comprising at least one ring.

12. The ergonomic medical instrument of claim 11, wherein the first support unit comprises two rings.

13. The ergonomic medical instrument of claim 1, wherein the second support unit is a ring.

14. A set of support units for a medical instrument comprising:
(a) a first support unit shaped to support at least one finger of a user, the first support unit comprising a first instrument engaging portion configured to be engaged with a first arm of the medical instrument and
(b) a second support unit shaped to support a thumb of a user, the second support unit comprising a second instrument engaging portion configured to be engaged with a second arm of the medical instrument.

15. The support units of claim 14, wherein the first support unit comprises two rings and wherein the second support unit comprises one ring.

16. The support units of claim 14, wherein the first support unit comprises two curves defining two seats and wherein the second support unit comprises one curve defining one seat.

17. The support units of claim 14, wherein the first instrument engaging portion is a clip and wherein the second engagement portion is a clip.

18. The support units of claim 14, wherein the first support unit and second support unit are selectively removable from the medical instrument.

19. The support units of claim 14, wherein the medical instrument is forceps with exchangeable tips.

20. A set of ergonomic medical instrument supports comprising:
(a) a first winged support comprising:
(i) a first curve defining a first seat;
(ii) a second curve defining a second seat;
(iii) a first ridge disposed between the first curve and the second curve; and
(iv) a first instrument engaging portion shaped for engagement with a first arm of an instrument and
(b) a second winged support unit comprising:
(i) a third curve defining a third seat;
(ii) a second ridge disposed centrally on the third curve; and
(iii) a second instrument engaging portion shaped for engagement with a second arm of the instrument.
